(19) United States
(12) Patent Application Publication
(10) Pub. No.: US 2025/0007190 A1
(43) Pub. Date: Jan. 2, 2025

(54) CLAMPING DEVICE AND SENSOR CABLE

(71) Applicant: Drägerwerk AG & Co. KGaA, Lübeck (DE)

(72) Inventor: Thomas GRAßL, Lübeck (DE)

(21) Appl. No.: 18/756,045

(22) Filed: Jun. 27, 2024

(30) Foreign Application Priority Data
Jun. 30, 2023 (DE) 10 2023 117 378.2

(51) Int. Cl. H01R 11/24

(52) U.S. Cl. CPC H01R 11/24

(57) ABSTRACT

A clamping device and a sensor cable with such a clamping device. The clamping device includes a holding element (30) with a holding surface (31), and a counter-holding element (40) with a counter-holding surface (41). The holding surface (31) and the counter-holding surface (41) are arranged to clampingly receive the electrical contact (20) therebetween. The holding element (30) is elastically deformable by actuation with an actuating force (F, F2) in such a way that a position of the holding surface (31) relative to the counter-holding surface (41) is variable between a first position (p1) for clamping reception of the electrical contact (20) and a second position (p2) for release of the electrical contact (20).

### CROSS REFERENCE TO RELATED APPLICATIONS

This application claims the benefit of priority under 35 U.S.C. § 119 of German Application 10 2023 117 378.2, filed Jun. 30, 2023, the entire contents of which are incorporated herein by reference.

### TECHNICAL FIELD

The present invention relates to a clamping device and a sensor cable with such a clamping device.

### BACKGROUND

It is known to electrically connect cables to an electrical contact, such as that of an electrode, via a clamping device. For this purpose, conventional electrodes normally have an electrically conductive stud (rivet) to which the clamping device can be connected. Another end of the cable is designed, for example, to connect to a diagnostic device, such as a patient monitor, an ECG device, an EEG device or the like, and has a plug for this purpose, for example. By means of the diagnostic device, a potential difference between two electrodes can be detected, for example, and can be visually and/or acoustically perceived by an operator of the diagnostic device.

A clamping device of the same type is known from the publication EP 3 797 688 A1. This clamping device is designed such that by pressing on both sides against an outer frame surface on a first frame axis, it is possible to effect a relative movement of a counter-holding device to a contact device into a first release position for releasing the electrical contact from the receiving space, and such that by pressing on both sides against the outer frame surface on a second frame axis different from the first frame axis, it is possible to effect a relative movement of the counter-holding device to the contact device into a second release position different from the first release position for releasing the electrical contact from the receiving space.

Compared to other clamping devices known from the publications U.S. Pat. No. 9,226,680 B, DE 196 43 988 C1, U.S. Pat. Nos. 4,040,697 A, 6,397,439 B, DE 40 09 938 A1, US 2011 151 728 A and US 2004 203 273 A, this clamping device has advantageously reduced manufacturing costs and improved handling. However, the handling of this clamping device, although improved, cannot be intuitively grasped by every operator.

### SUMMARY

It is an object of the invention to provide a clamping device and a corresponding sensor cable which do not have at least some of the disadvantages of the aforementioned clamping devices. In particular, the present invention is therefore based on the object of providing a clamping device and a corresponding sensor cable whose handling is further improved.

These and other objects are attained by a clamping device with clamping device features according to the invention and by a sensor cable with sensor cable features according to the invention.

Further features and details of the invention are provided by this disclosure including the claims, the description, and the figures.

Features and details that are described in connection with the clamping device according to the invention also apply in connection with the sensor cable according to the invention and vice versa, so that reference can always be made reciprocally with regard to the disclosure of the individual aspects of the invention.

According to the invention, a clamping device for electrically connecting a cable to an electrical contact is provided. The clamping device has a holding element (retaining element) with a holding surface and a counter-holding element with a counter-holding surface. The holding surface and the counter-holding surface are configured to clamp the electrical contact between them. The holding element can be elastically deformed by an actuation, in particular by an actuation pressing on the holding element, with an actuation force such that a position of the holding surface relative to the counter-holding surface can be changed between a first (closed) position for clamping reception of the electrical contact and a second (open) position for release of the electrical contact. The holding element has a snap point, so that when the holding element is actuated, a restoring force counteracting the actuating force increases until the snap point is reached, whereby when the snap point is reached, the holding surface abruptly changes from the first position to the second position and the restoring force drops.

In other words, the present invention provides a clamping device in which the holding surface and the counter-holding surface are changeable in position relative to each other by a cracking frog (clicker) mechanism. In still other words, a change between the first position and the second position consequently occurs abruptly, i.e. snappingly, upon reaching a certain actuating force which characterizes the snap point. These effects of snapping or the abrupt change in position of the holding surface formed by the holding element when actuated with sufficient actuating force up to the snap point and the sudden drop in the restoring force of the holding element acting against the actuating force are known as the “cracking frog” effect (snap action effect or clicker effect), which is provided by the cracking frog mechanism or clicker mechanism according to the invention.

The “cracking frog” effect, which can be perceived at least haptically and preferably also acoustically by the operating person when the holding element is deformed, thus provides feedback about reaching the snap point and therefore about the opening of the clamping device, i.e. a change from the first position to the second position. The operator thus receives immediate feedback that the clamping device has opened. Any uncertainties as to whether the actuating force applied is sufficient to open the clamping device can thus be eliminated. After the holding element has been released, the closing of the clamping device, i.e. a change from the second position to the first position, can also be perceived haptically and possibly also acoustically.

The present invention is thus improved over known clamping devices in particular in that a comparatively uniform force/displacement curve is produced during actuation in the case of conventional clamping devices, i.e. a somewhat “analog” actuation occurs. In contrast, the provision of a clamping device with a click-frog effect according to the invention allows a “digital” actuation to a certain extent. This enables the operator to clearly distinguish between two actuation states, namely the states “open”, i.e. that the second position is present, and “closed”, i.e. that the first position is present. Unwanted intermediate positions can be avoided.

A further advantage of the provision of the cracking frog effect according to the invention is that once the snap point has been exceeded, only a small restoring force acts against the actuating force, so that the operator can hold the holding element in the open state with little effort for attachment to the electrical contact or for removal from the electrical contact. On average over time, this reduces the strain on the operator compared to known clamping devices.

The present invention is not limited to the field of medical technology, is applicable in all technical fields in which a clamping holding of an electrical contact is desired. A further field of application arises, for example, in measurement technology for connecting an electrical contact to an electrical measuring device.

A holding element is understood to be a part of the clamping device that provides a holding surface, i.e. a surface that is suitable for, preferably rear-engaging, contact with or around the electrical contact.

A counter-holding element is understood to be a part of the clamping device which provides a counter-holding surface, i.e. a surface which is suitable for, preferably rear-engaging, contact with or around the electrical contact.

The holding element and the counter-holding element can be provided as separate components of an assembly, which are connected to form the clamping device. Alternatively, the holding element and the counter-holding element can be configured as respective areas of an integrally configured (one piece) clamping device.

For example, the holding element may consist of, or comprise, an elastically deformable, i.e. soft, structure that can be subjected to an actuating force that can be applied by an operator, or comprise such a structure and be connected to a counter-holding element that is essentially rigid with respect to an operating force that can be applied by an operator in a multi-component injection molding process. In a further variant, the counter-holding element can be provided essentially rigid and, after being connected to a flat spring, such as a spring plate, can be overmolded together with a soft component in an injection molding process. In yet another variant, a cable can first be connected to the holding element, preferably its flat spring, if present, and then, in a subsequent step, the holding element and the cable can be at least partially overmolded with the soft component as strain relief in an injection molding process.

A clamped reception of the electrical contact between the holding surface and the counter-holding surface refers to a force-fit and/or form-fit reception of the electrical contact between the holding surface and the counter-holding surface.

Deformability by actuation with an actuating force refers to an elastic deformability of the holding element, which can be achieved by an actuating force applied by an operator. The force applied by the operator is understood as the actuating force.

A snap point is understood to be the deformation state of the holding element at which the holding surface abruptly changes from the first position to the second position and the restoring force counteracting the actuating force drops.

Preferably, the holding element has a flat spring that provides the snap point of the holding element.

In this way, the elastic deformation behavior of the holding element can be adapted in a particularly targeted manner.

A flat spring is a spring that is configured as a bending spring made from an essentially flat starting material. The flat spring can have a spatial extension or segments with spatial extension as a result of forming steps, whereby such a spring is also referred to as a flat-form spring. Examples of such forming steps are embossing, bending and/or punching. An example of a flat spring is a spring sheet (spring plate) or a leaf spring. It is not necessary for the flat spring to be made of a metallic material. The flat spring can also be made of plastic.

Preferably, the holding element, in particular the flat spring, has a stable state, which corresponds to the first position of the holding surface, and also has a metastable or unstable state, which corresponds to the second position of the holding surface.

In this way, it can be advantageously ensured that the holding element or the flat spring snaps back from the open state (second position) into the stable closed state (first position) after the load has been removed, i.e. after the actuating force has been removed. Compared to known holding elements or flat springs, which are bistable, the closing process of the holding element or flat spring can thus be improved.

Preferably, the holding element, in particular the flat spring, has an arcuate (curved) segment with an arcuate shape, whereby the segment is configured in such a way that it can be bent through the snap point against the arcuate shape by the actuating force and snaps back into the original shape through the snap point when the actuating force is removed due to a restoring force of the holding element, in particular the flat spring.

It is therefore intended that the drop in the restoring force of the holding element, in particular the flat spring, is caused by a correspondingly sudden change in shape. According to further preferred teachings, the change in shape can be accompanied by an audible cracking (clicking) sound.

Preferably, the segment is curved outwards in relation to the clamping device in the stable state, and the segment is arranged to curve inwards in relation to the clamping device when changing from the first position to the second position.

The preferred abrupt change in shape can therefore be achieved by the holding element, in particular the flat spring, being convexly curved outwards in the area of the segment in the longitudinal direction against the direction of actuation. The holding element, in particular the flat spring, can therefore be curved outwards in a trough-like manner in the longitudinal direction. When the holding element is actuated, the trough-like preform must therefore first be pressed through to the snap point, which requires an initial, comparatively high actuating force. Once the snap point has been passed, the actuating force is only countered by a restoring force corresponding to a spring force of the holding element, in particular the flat spring, so that the operator only has to apply a second, comparatively low actuating force for further deformation or to maintain the deformation.

It is preferable that the maximum deformation path of the holding element or the flat spring that can be achieved by actuating force is limited by a stop. Overstretching of the holding element or the flat spring can thus be avoided.

Preferably, the holding element, in particular the flat spring, comprises an electrically conductive material or consists of an electrically conductive material, whereby the holding element, in particular the flat spring, provides an electrical interface between the electrical contact and the cable.

Particularly preferably, the holding element itself or the flat spring can also act as an electrical conductor, so that this functional integration can advantageously reduce the number of components required to provide the clamping device and consequently the manufacturing costs.

An electrically conductive material can, for example, be a metal such as a steel, particularly preferably a spring steel, most preferably a spring steel according to EN 10089. The flat spring is particularly preferably configured as a spring plate.

As described above, the spring plate can have segments with a spatial extension or be configured spatially as a whole. In particular, the spring plate can have an arcuate segment as described above.

By providing the flat spring as a spring plate, the manufacturing costs of the clamping device can be advantageously reduced, as spring plates are available at low cost.

Preferably, the holding element is connected to the counter-holding element in bearings in two spaced-apart areas.

In this way, an actuating surface for actuation with the actuating force can be provided between the spaced-apart areas and force absorption of the actuating force by the bearing points can be ensured.

A bearing connection can be configured as a fixed bearing and/or a floating bearing. It is preferable to provide two fixed bearings, as this is possible without a gap. This in turn is advantageous with regard to the cleanability of the clamping device.

Preferably, the holding surface and the counter-holding surface are configured to grip the electrical contact in a gripping plane, wherein the actuating force can be applied to the holding element in an actuating direction, and wherein the actuating direction is parallel to the gripping plane.

In other words, the electrical contact is gripped laterally. This ensures that when the clamping device is attached and detached from the electrical contact, no or only low pressure forces are exerted on the electrical contact outside the gripping plane, which could be perceived as uncomfortable by a patient (associated with the electrical contact, such as a contact of an electrode applied to the patient). Consequently, patient comfort can be improved by such a clamping device.

In a preferred embodiment, the holding surface and the counter-holding surface are set up to grip the electrical contact in a plurality of gripping planes, whereby the actuating force can be applied to the holding element in the actuating direction, and whereby the actuating direction is parallel to the plurality of gripping planes.

This means that there is no need to change the grip if the clamp is gripped “incorrectly”, as it can grip on both sides.

According to the invention, a sensor cable is further provided for electrically connecting an electrical contact to a medical device, the sensor cable comprising a number of cables (one or more cables—a cable arrangement) and a number of clamping devices (one or more clamping devices).

In this respect, a number is understood to be a singular or a plural number. In particular, the cable may be one or more cables and is also referred to as a cable arrangement. The sensor cable (comprising a cable arrangement with one or more cables) may also comprise one or more clamping devices.

Each cable can have one or more poles.

Preferably, the number of cables and the number of clamping devices are integrally formed with one another. In particular, the cable arrangement and the clamping device (or clamping devices) may be integrally formed with each other as a one piece structure.

These features and further details of the invention are also apparent from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1a is a side view showing an embodiment of a clamping device according to the invention in a closed state;

FIG. 1b is a side view showing the clamping device according to the invention according to FIG. 1a in an open state;

FIG. 1c is a perspective view showing the clamping device according to the invention according to FIG. 1a in the closed state;

FIG. 2 is a perspective view showing an embodiment of a flat spring according to the invention; and

FIG. 3 is a schematic displacement-restoring force diagram of a clamping device according to the invention.

### DESCRIPTION OF PREFERRED EMBODIMENTS

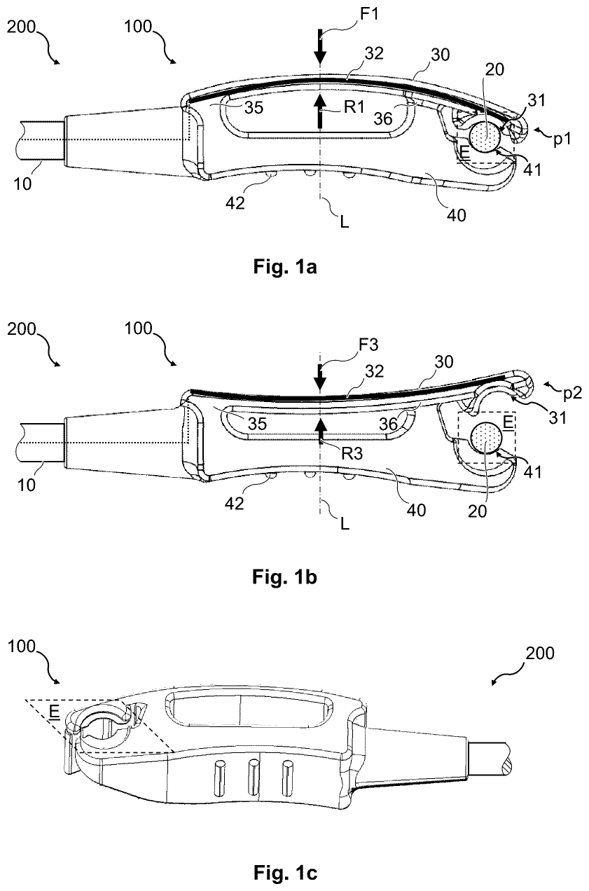

Referring to the drawings, according to the invention, a clamping device 100 is provided. An embodiment example of a clamping device 100 according to the invention is shown in FIGS. 1a-1c.

The clamping device 100 shown is used to electrically connect a cable (cable arrangement) 10 to an electrical contact 20. The clamping device 100 has a holding element 30 with a holding surface 31 and a counter-holding element 40 with a counter-holding surface 41. In the example shown, the holding element 30 and the counter-holding element 40 are provided joined together in a multi-component injection molding process.

The holding surface 31 and the counter-holding surface 41 are configured to clamp the electrical contact 20, shown as a schematic circle, between them. The electrical contact 20 can have an essentially arbitrary contour and spatial shape.

The holding element 30 can be elastically deformed by actuation with an actuating force F such that a position of the holding surface 31 relative to the counter-holding surface 41 can be varied between a first position p1 for clamping reception of the electrical contact 20 and a second position p2 for release of the electrical contact 20.

In this respect, a closed state of the clamping device 100 is shown in FIG. 1a, i.e. a state in which the position of the holding surface 31 relative to the counter-holding surface 41 is the first position p1.

The holding element 30 has a snap point, so that when the holding element 30 is actuated, a restoring force R counteracting the actuating force F increases until the snap point is reached and, when the snap point is reached, the holding surface 31 abruptly changes from the first position p1 to the second position p2 and the restoring force R drops.

In this respect, an open state of the clamping device 100 is shown in FIG. 1b, i.e. a state in which the position of the holding surface 31 relative to the counter-holding surface 41 is the second position p2.

If an actuating force F1 is applied to the holding element 30 in the closed state, which is less than the actuating force F2 corresponding to the snap point, only a slight deformation of the holding element 30 takes place without opening the clamping device. Only when the actuating force F2 reaches the actuating force F2 corresponding to the snap point does an abrupt change from the first position p1 to the second position p2 take place, i.e. a change from the state shown in FIG. 1a to the state shown in FIG. 1b. Maintaining this open state requires only a comparatively low actuating force F3, i.e. an actuating force F3 that is lower than the actuating force F2 corresponding to the snap point.

This behavior of the clamping device and cable according to the invention is illustrated in an abstracted representation of the principle in the diagram according to FIG. 3, in which the deformation path (or also the actuation path) s of the holding element 30 is schematically plotted against the restoring force R of the holding element 30. A relatively high actuating force must be applied up to the snap point, which is characterized by a predetermined restoring force R2 with a predetermined deformation path s2, in order to deform the holding element 30. A deformation path s1 with an actuating force F1 corresponding to the restoring force R1 therefore does not yet trigger a snap or an abrupt change in shape. When the snap point is reached with the deformation path s2 and with an actuating force F2 corresponding to the restoring force R2, an abrupt deformation of the holding element 30 takes place, whereby the deformation path s abruptly increases to s3 and at the same time the restoring force R3 and thus the actuating force F3 required to maintain the deformation state decreases (drops).

The holding element 30 can have a stable state, which corresponds to the first position p1 of the holding surface 31, and can also have a metastable or unstable state, which corresponds to the second position p2 of the holding surface 31.

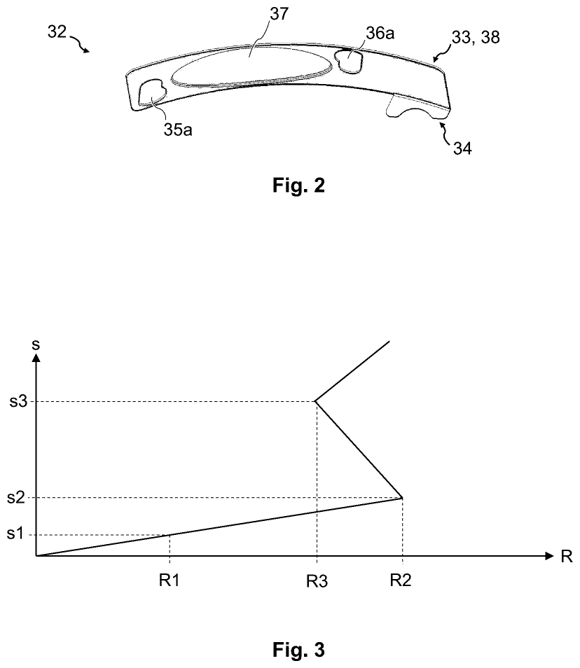

In the embodiment example shown, the holding element 30 can have a flat spring 32, which provides the snap point s2, F2 of the holding element 30. In the embodiment example shown, the flat spring 32 is configured as a spring plate 32 with spatially extending segments, as shown in FIG. 2. The flat spring 32 thus has an arcuate segment 33 with an arcuate shape. In the embodiment example shown, the flat spring 32 is formed in its longitudinal direction by the arcuate segment 33. The segment 33 is configured in such a way that it can be bent by the actuating force F2 against the arcuate shape through the snap point and snaps back into the original shape when the actuating force F2 is removed due to the restoring force of the flat spring 32 through the snap point.

It is not necessary for the flat spring 32 to have the arcuate segment 33. For example, the holding element 30 itself, molded from plastic, can have an arcuate segment 33 with an arcuate shape, whereby the segment 33 can be configured in such a way that it can be bent by the actuating force F against the arcuate shape through the snap point s2, F2 and, when the actuating force F is removed, snaps back into the original shape through the snap point s2, F2 due to a restoring force of the holding element 30.

The segment 33 according to FIG. 2 is curved outwards in relation to the clamping device 100 in the stable state and is set up to curve inwards in relation to the clamping device 100 when changing from the first position p1 to the second position p2, as can be seen, for example, in the comparison of FIG. 1a to FIG. 1b.

In the illustrated embodiment example, the flat spring 32 may comprise an electrically conductive material or consist of an electrically conductive material. One example of such a material is spring steel.

As can be seen in FIGS. 1a and 1b, the flat spring 32 can provide an electrical interface 38 between the electrical contact 20 and the cable 10.

However, the electrically conductive material does not have to be provided by the flat spring 32. Thus, it is possible that the holding element 30 comprises an electrically conductive material or consists of an electrically conductive material, and that the holding element 30 provides the electrical interface 38 between the electrical contact 20 and the cable 10.

As further shown in FIGS. 1a and 1b, the holding element 30 can be connected to the counter-holding element 40 in a bearing manner in two spaced-apart regions, namely in a first bearing region 35 and in a second bearing region 36. For this purpose, the flat spring 32, as shown in FIG. 2, can have two, for example, stamped or embossed areas, which correspondingly provide a first bearing section 35a and second bearing sections 36a.

As shown in FIG. 2, the flat spring 32 can also have a forming section (shaping section) 37 in order to further adapt the mechanical properties of the flat spring 32.

As can be seen in FIGS. 1a-1c, the holding surface 31 and the counter-holding surface 41 are configured to grip the electrical contact 20 in a gripping plane E, i.e. to perform a closing or opening movement in the gripping plane E. In FIGS. 1a, 1b, the gripping plane E lies in the viewing plane; in FIG. 1c, the gripping plane E extends into the viewing plane.

The actuating force F can be applied to the holding element 30 in an actuating direction L, whereby the actuating direction L is parallel to the gripping plane E.

To grip the electrical contact 30, the flat spring 32 can, for example, have a gripping segment 34, which can extend away from the arcuate segment 33 at an angle. In the preferred case that the flat spring 32 comprises an electrically conductive material or consists of an electrically conductive material, the gripping segment 34 simultaneously forms an electrical contacting surface of the holding surface 31.

As can be seen particularly clearly in FIG. 1c, the counter-holding surface 41 can also have a shape that enables or improves the gripping of the electrical contact 30.

As can be seen from FIGS. 1a-1c, a sensor cable 200 for electrically connecting an electrical contact 20 to a medical device is also provided according to the invention. The sensor cable 200 has a number of (one or more) cables 10 and a number of (one or more) clamping devices 100 as described above.

The number of cables 10 and the number of clamping devices 100 can be formed integrally with one another.

While specific embodiments of the invention have been shown and described in detail to illustrate the application of the principles of the invention, it will be understood that the invention may be embodied otherwise without departing from such principles.

### LIST OF REFERENCE SYMBOLS

10 Cable-cable arrangement
20 Electrical contact
30 Holding element
31 Holding surface
32 Flat spring, spring plate
33 Arcuate (curved) segment
34 Gripping segment
35 First bearing area
35a First bearing section
36 Second bearing area
36a Second bearing section
37 Forming section (shaping section)
38 Electrical interface
40 Counter-holding element
41 Counter-holding surface
42 Surface structure
100 Clamping device
200 Sensor cable
E Gripping plane
F1, F3 Actuating force
R, R1, R2, R3 Restoring force
L Actuating direction
p1 First position
p2 Second position
s, s1, s2, s3 deformation path (or also actuation path)

What is claimed is:

1. A clamping device for electrically connecting a cable to an electrical contact, the clamping device comprising:
a holding element with a holding surface; and
a counter-holding element with a counter-holding surface,
wherein the holding surface and the counter-holding surface are configured to clampingly receive the electrical contact therebetween,
wherein the holding element is elastically deformable by actuation with an actuating force to vary a position of the holding surface relative to the counter-holding surface between a first position for clamping reception of the electrical contact and a second position for release of the electrical contact,
wherein upon actuation of the holding element with an actuation force, a restoring force counteracting the actuating force increases until a snap point is reached, and
wherein upon the snap point being reached, the holding surface abruptly changes from the first position to the second position and the restoring force decreases.

2. A clamping device according to claim 1, wherein the holding element comprises a flat spring configured to provide the snap point of the holding element.

3. A clamping device according to claim 1,
wherein the holding element has a stable state which corresponds to the first position of the holding surface, and
wherein the holding element has a metastable or unstable state which corresponds to the second position of the holding surface.

4. A clamping device according to claim 1,
wherein the holding element has an original shape with an arcuate segment with an arcuate shape,
wherein the arcuate segment is configured such that the arcuate segment is bendable in a direction against the arcuate shape through the snap point by the actuating force and snaps back into the original shape through the snap point due to the restoring force, upon the actuating force being removed.

5. A clamping device according to claim 4,
wherein the arcuate segment is curved outwards with respect to the clamping device in the stable state, and
wherein the arcuate segment is configured to curve inwards with respect to the clamping device upon changing from the first position to the second position.

6. A clamping device according to claim 1,
wherein the holding element comprises an electrically conductive material, and
wherein the holding element provides an electrical interface between the electrical contact and the cable.

7. A clamping device according to claim 2, wherein the flat spring is configured as a spring plate.

8. A clamping device according to claim 1, wherein the holding element is connected to the counter-holding element to bear in two regions spaced apart from one another.

9. A clamping device according to claim 1,
wherein the holding surface and the counter-holding surface are configured to grip the electrical contact in a gripping plane,
wherein the actuating force is applied to the holding element in an actuating direction, and
wherein the actuating direction is parallel to the gripping plane.

10. A sensor cable for electrically connecting an electrical contact to a medical device, sensor cable comprising:
a cable arrangement comprising one or more cables; and
a clamping device connected to the cable arrangement, the clamping device comprising a holding element with a holding surface and a counter-holding element with a counter-holding surface,
wherein the holding surface and the counter-holding surface are configured to clampingly receive the electrical contact therebetween,
wherein the holding element is elastically deformable by actuation with an actuating force to vary a position of the holding surface relative to the counter-holding surface between a first position for clamping reception of the electrical contact and a second position for release of the electrical contact,
wherein upon actuation of the holding element with an actuation force, a restoring force counteracting the actuating force increases until a snap point is reached, and
wherein upon the snap point being reached, the holding surface abruptly changes from the first position to the second position and the restoring force decreases.

11. A sensor cable according to claim 10, wherein the cable arrangement and the clamping device are integrally formed with each other as a one piece structure.

12. A sensor cable according to claim 10, wherein the holding element comprises a flat spring configured to provide the snap point of the holding element.

13. A sensor cable according to claim 10,
wherein the holding element has a stable state which corresponds to the first position of the holding surface, and
wherein the holding element has a metastable or unstable state which corresponds to the second position of the holding surface.

14. A sensor cable according to claim 10,
wherein the holding element has an original shape with an arcuate segment with an arcuate shape,
wherein the arcuate segment is configured such that the arcuate segment is bendable in a direction against the arcuate shape through the snap point by the actuating force and snaps back into the original shape through the snap point due to the restoring force, upon the actuating force being removed.

15. A sensor cable according to claim 14,
wherein the arcuate segment is curved outwards with respect to the clamping device in the stable state, and
wherein the arcuate segment is configured to curve inwards with respect to the clamping device upon changing from the first position to the second position.

16. A sensor cable according to claim 10,
wherein the holding element comprises an electrically conductive material, and
wherein the holding element provides an electrical interface between the electrical contact and the cable.

17. A sensor cable according to claim 12, wherein the flat spring is configured as a spring plate.

18. A sensor cable according to claim 10, wherein the holding element is connected to the counter-holding element to bear in two regions spaced apart from one another.

19. A sensor cable according to claim 10,
wherein the holding surface and the counter-holding surface are configured to grip the electrical contact in a gripping plane,
wherein the actuating force is applied to the holding element in an actuating direction, and
wherein the actuating direction is parallel to the gripping plane.
